(19) United States
(12) Patent Application Publication
(10) Pub. No.: US 2008/0319505 A1
(43) Pub. Date: Dec. 25, 2008

(54) Integrated Transcranial Current Stimulation and Electroencephalography Device

(75) Inventors: Edward S. Boyden, Cambridge, MA (US); Zackary M. Anderson, Beverly Hills, CA (US); Ekavali Mishra, Washington, DC (US)

Correspondence Address: NORMA E HENDERSON;HENDERSON PATENT LAW, 13 JEFFERSON DR, LONDONDERRY, NH, 03053, US

(73) Assignee: Massachusetts Institute of Technology, Cambridge, MA (US)

(21) Appl. No.: 12/118,092

(22) Filed: May 9, 2008

Related U.S. Application Data
(60) Provisional application No. 60/916,953, filed on May 9, 2007.

(51) Int. Cl. A61N 1/04; A61B 5/0476

(52) U.S. Cl. 607/45; 600/544

(57) ABSTRACT

In an integrated transcranial current stimulation and electroencephalography device, data obtained by EEG electrodes is received, amplified, converted, and then processed by a microcontroller that extracts frequency information from the sampled data and produces signals in response to the extracted EEG data. These signals are converted to create a software-definable alternating voltage used to control a current-source that connects to stimulation electrodes. The device may further download recorded EEG data from the microcontroller to a computer for further analysis. Using the device, EEG signals are detected, then received, processed, and analyzed by a microcontroller to identify the patient state. Based on the patient state and the desired protocol, a type and amount of current stimulation to apply to the patient is determined and a control signal is sent from the microcontroller to the current source in order to trigger the transcranial current stimulation of the patient.

### RELATED APPLICATIONS

This application claims the benefit of U.S. Provisional Application Ser. No. 60/916,953, filed May 9, 2007, the entire disclosure of which is herein incorporated by reference.

### FIELD OF THE TECHNOLOGY

The present invention relates to devices used in electroencephalography and, in particular, to an integrated device for electroencephalography with transcranial current stimulation.

### BACKGROUND

Noninvasive, low-frequency oscillatory current stimulation, including direct current stimulation, of the brain through the skull has been shown to improve cognitive functions. In order to accomplish optimal behavioral outcomes, however, such currents must be computed in response to, and delivered during the presence of, appropriate brain states. Studies have demonstrated that low-frequency oscillatory currents, or even direct current (DC), applied to the brain can enhance memory [L. Marshall, H. Helgadottir, M. Molle et al. “Boosting slow oscillations during sleep potentiates memory”, Nature. 2006 Nov. 30; 444(7119):610-3; L. Marshall, M. Molle, M. Hallschmid et al. “Transcranial direct current stimulation during sleep improves declarative memory”, J Neurosci. 2004 Nov. 3; 24(44):9985-92; F. Fregni, P. S. Boggio, M. Nitsche et al., “Anodal transcranial direct current stimulation of prefrontal cortex enhances working memory”, Experimental brain research. Experimentelle Hirnforschung. 2005 September; 166(1):23-30; P. S. Boggio, R. Ferrucci, S. P. Rigonatti et al. “Effects of transcranial direct current stimulation on working memory in patients with Parkinson's disease”, Journal of the neurological sciences. 2006 Nov. 1; 249(1):31-8;] reduce pain [F. Fregni, P. S. Boggio, M. C. Lima et al. “A sham-controlled, phase II trial of transcranial direct current stimulation for the treatment of central pain in traumatic spinal cord injury”, Pain. 2006 May; 122(1-2):197-209], and improve the symptoms of Parkinson's disease [P. S. Boggio, R. Ferrucci, S. P. Rigonatti et al. “Effects of transcranial direct current stimulation on working memory in patients with Parkinson's disease”, Journal of the neurological sciences. 2006 Nov. 1; 249(1):31-8; F. Fregni, P. S. Boggio, M. C. Santos et al. “Noninvasive cortical stimulation with transcranial direct current stimulation in Parkinson's disease”, Mov Disord. 2006 October; 21(10):1693-702] and stroke [F. Hummel, P. Celnik, P. Giraux et al. “Effects of non-invasive cortical stimulation on skilled motor function in chronic stroke”, Brain. 2005 March; 128(Pt 3):490-9; F. Hummel and L. G. Cohen. “Improvement of motor function with noninvasive cortical stimulation in a patient with chronic stroke”, Neurorehabilitation and neural repair. 2005 March; 19(1):14-9]. The method is safe and easy to use [M. A. Nitsche, D. Liebetanz, N. Lang et al., “Safety criteria for transcranial direct current stimulation (tDCS) in humans”, Clin Neurophysiol. 2003 November; 114(11):2220-2].

Although transcranial current stimulation (TCS) may result in changes in the electroencephalograph (EEG) signal [A. Antal, E. T. Varga, T. Z. Kincses et al. “Oscillatory brain activity and transcranial direct current stimulation in humans”, Neuroreport. 2004 Jun. 7; 15(8):1307-10], TCS itself has almost always been used in an open loop fashion, with delivery occurring without conditioning on any particular EEG signature, and without use of the subsequently measured EEG signal to modify the stimulation. The need for a linked EEG-TCS machine has grown increased in recent years, as preliminary data has shown that current stimulation at a particular frequency (0.75 Hz) upon observation of a particular EEG signature (the onset of slow-wave sleep) can enhance memory in a biophysically plausible way [L. Marshall, H. Helgadottir, M. Molle et al. “Boosting slow oscillations during sleep potentiates memory”, Nature 444. 2006 Nov. 30; 444(7119):610-3]. Thus, by inducing a potential at the proper frequency and right time, a number of illnesses, such as Alzheimer's, may be treatable, at least at the symptom level, in a patient-customized and focused way. In addition, such a device may be used for consumer-targeted memory augmentation applications, as well as enabling a wide variety of treatments for disorders ranging from epilepsy to stroke to Parkinson's disease.

Traditional electroencephalogram (EEG) techniques to determine sleep stages require large, expensive machinery and experienced analysts to decipher the data. Indeed, sleep scoring is still primarily done by human observation. In addition, non-invasive brain stimulation has typically relied on custom-tuned machinery that is simply not practical for consumers to use without the assistance of healthcare professionals. There is no device that has combined these two powerful technologies, nor have they been combined in a small, portable, inexpensive format.

### SUMMARY

The present invention is an integrated, wearable, noninvasive device for detecting brain states via electroencephalography and transcranially delivering current of appropriate spectral properties and amplitude to targeted locations on the skull surface for brain stimulation. The present invention employs a single microcontroller and associated hardware that digitizes EEG data, analyzes the features of the extracted data, and, based on the analysis, controls further transcranial stimulation of the patient.

In one aspect of the present invention, data obtained by the EEG electrodes is received, amplified, converted, and then processed by a microcontroller that extracts frequency information from the sampled data. The data is analyzed to identify a patient state and, based on that state and the intended result, a desired transcranial current stimulation amount and type is determined. The microcontroller then produces control signals that are converted to create a software-definable alternating voltage used to control a current-source that connects to the stimulation electrodes. The device may optionally download recorded EEG data from the microcontroller to a computer for further analysis.

In another aspect, the present invention enables the use of many different protocols to achieve the desired result. In a preferred embodiment, the EEG is linear filtered in order to emphasize a desired EEG feature or pattern of activity. This filtered version is then sent back to the patient through the TCS, thus amplifying that specific frequency in the brain. Other useful protocols include, but are not limited to, a phase-estimator algorithm, which permits amplification of brain waves with real-time control, a protocol that provides patient-customized bandwidth, and a protocol designed to continuously entrain the brain's oscillations through multiplex reading from and writing to the brain.

### BRIEF DESCRIPTION OF THE DRAWINGS

Other aspects, advantages and novel features of the invention will become more apparent from the following detailed description of the invention when considered in conjunction with the accompanying drawings wherein:

FIG. 1 is a block diagram of an embodiment of an integrated device for transcranial current stimulation and electroencephalography according to the present invention;

FIG. 2 is a schematic diagram depicting an embodiment of operational amplifiers with automatic gain control used to amplify the electrodes used for EEG, according to one aspect of the present invention;

FIG. 3 is a schematic diagram depicting an embodiment of a digital signal processing microcontroller with analog-to-digital converters used to extract frequency information from the sampled data, according to another aspect of the present invention;

FIG. 4 is a schematic diagram depicting an embodiment of digital-to-analog converters used to create a software-definable alternating voltage, and a voltage-controlled-current-source which connects to the stimulation electrodes, according to another aspect of the present invention;

FIG. 5 is an exemplary board schematic for an embodiment of a device according to the present invention; and

FIG. 6 is a flowchart illustrating the operation of an embodiment of the present invention.

### DETAILED DESCRIPTION

The present invention is an integrated, wearable, noninvasive device capable of detecting brain states via electroencephalography (EEG) and delivering current of appropriate spectral properties and amplitude to targeted locations on the skull surface, with bi-directional control, for contextually-appropriate current brain stimulation. It is an integrated device that detects EEG signals and computes, through an algorithm, current or voltage signals to be delivered through electrodes on the scalp. The present invention is the first single-microcontroller solution that both digitizes EEG data and analyzes features thereof such as, but not limited to, spectral, time-series, or wavelets, and delivers currents of various patterns, including, but not limited to, DC, sinusoids of various frequency, and more complex signals, in a dynamic way based on the EEG readings.

In one aspect, the present invention is a small, portable, battery-powered device that automatically detects sleep cycles, can self-adjust its automatic detection algorithm to fit an individual's actual recorded data, records brain activity to local memory for later download to a computer, and stimulates the brain during pre-determined sleep cycles. In another aspect, the present invention is a method to enhance memory retention by inducing electrical oscillations across the brain during sleep and automatically detect sleep cycles using a “learning algorithm”.

In the simplest form, the device of the present invention waits until an EEG of a particular kind has appeared, and then delivers a pre-determined current stimulus. In a more complex implementation, the device delivers a current stimulus, the details of which are pre-computed dependent upon one or more features of the detected EEG signal. In a yet more complex implementation, the device continuously adapts the currents delivered to one or more electrodes on the surface of the scalp, as a function of current and past EEG features, and can also create a model of future EEG changes, the disagreement of which with acquired data may modify one or more aspects of the algorithm.

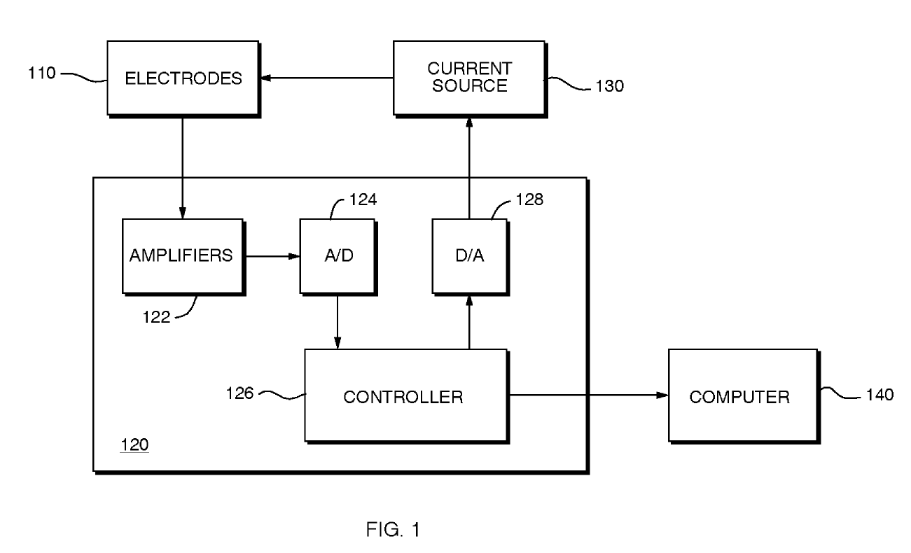

FIG. 1 is a block diagram of an embodiment of an integrated device for transcranial current stimulation and electroencephalography according to the present invention. In FIG. 1, data obtained by electrodes 110 used for EEG is received by integrated transcranial current stimulation and EEG device 120 is amplified by amplifiers 122, converted by A/D converters 124, then sent to digital signal processing (DSP) microcontroller 126 which extracts frequency information from the sampled data. Signals from microcontroller 126 in response to the extracted EEG data are converted by D/A converters 128 to create a software-definable alternating voltage used to control voltage-controlled-current-source 130 that connects to stimulation electrodes 110. Device 120 also downloads recorded EEG data from microcontroller 126 to computer 140 via a universal serial bus or wireless interface.

In a preferred embodiment, a circuit contains a microcontroller, optionally with additional on-board RAM or flash memory, which digitizes EEG data acquired through one or more ADCs (and optional upstream amplifier chains), and then spectrally processes the EEG data to determine which sleep cycle the subject is in. The device then induces a programmable, oscillating current through one or more output DACs (and optional downstream amplifiers) across the scalp, triggered by the occurrence of a particular spectral signature, the onset of the first epoch of slow-wave sleep in a sleep period. The device uses one or more gold-plated electrodes placed on the scalp (preferably with conductive gel) to read the EEG data, and also employs two separate electrodes to induce a current waveform when the microcontroller software determines that the spectral EEG condition is met. Alternatively, the same electrodes may be used to both detect EEG and deliver currents. A typical location of an EEG electrode might be the C3-A2 location, and typical locations of the current-delivering electrodes include the frontal and mastoid regions for the head. In one embodiment, electrode pairs are kept quite close, in order to cause currents to pass through desired local circuits in the brain.

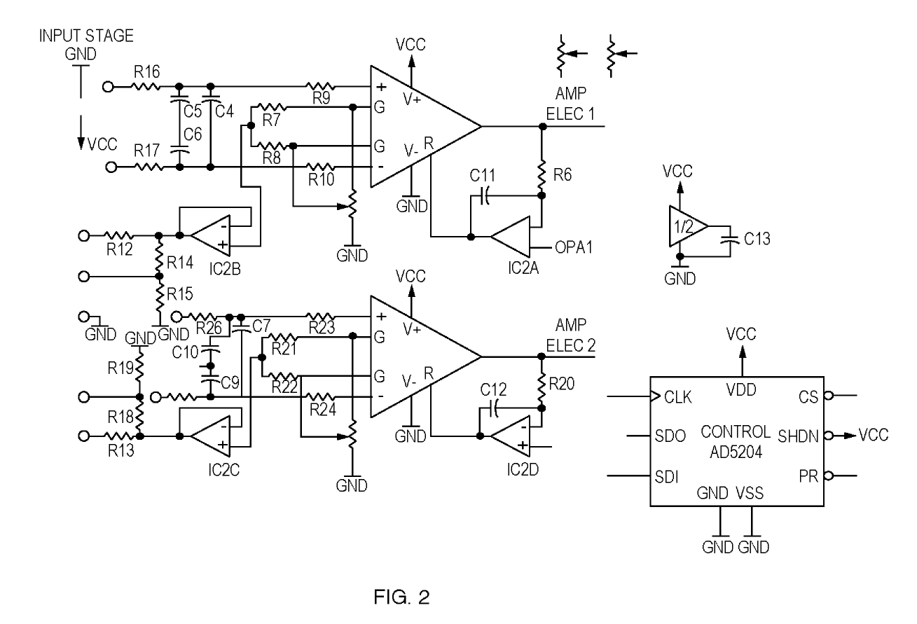

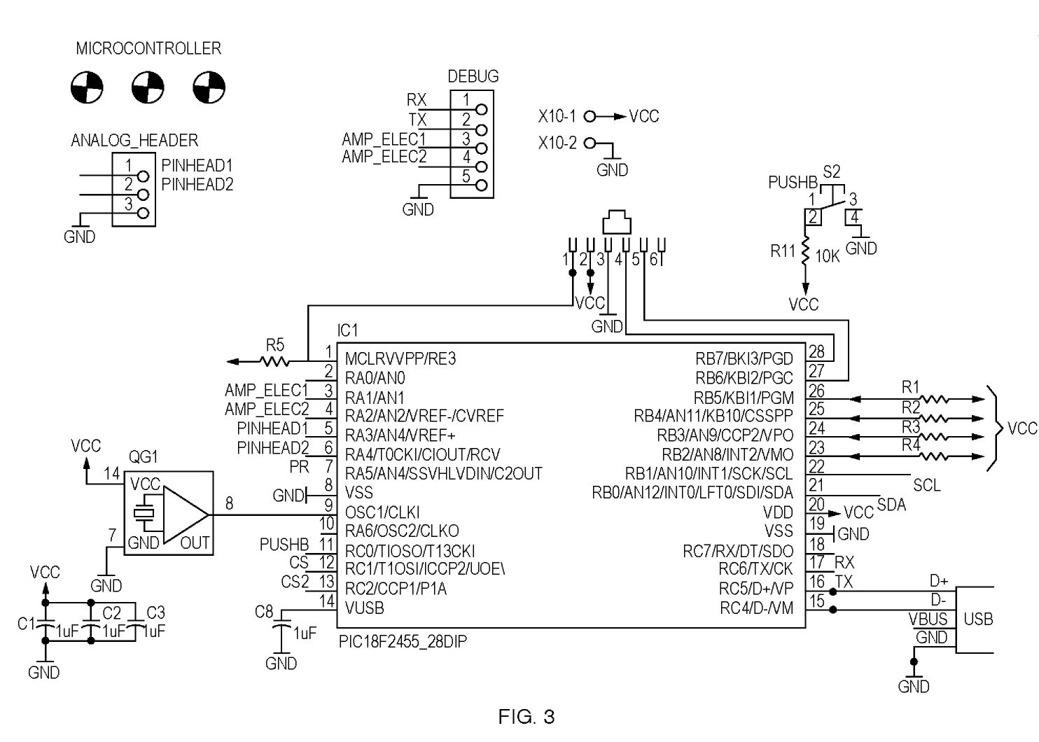

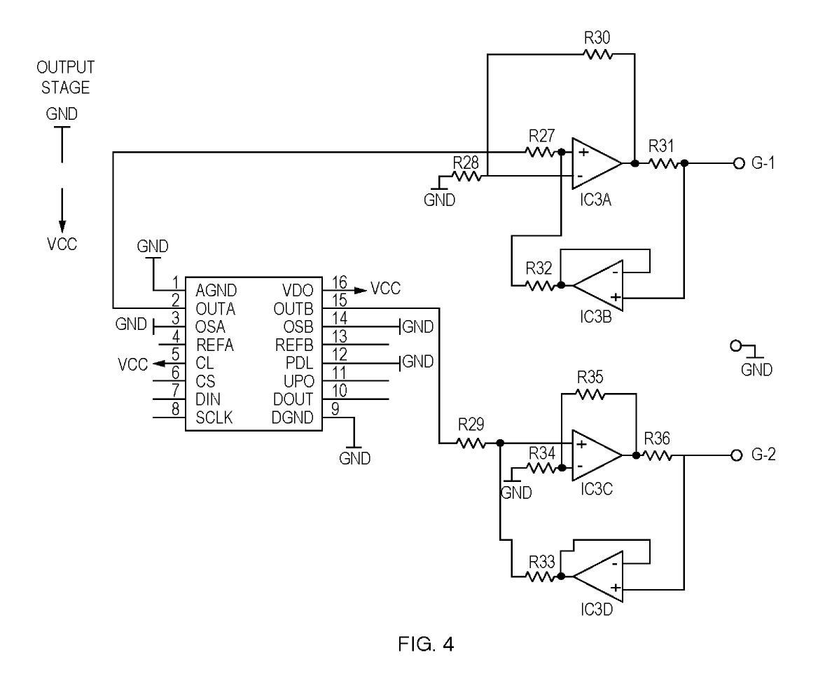

As depicted in FIGS. 2-4, the preferred embodiment comprises several operational amplifiers with automatic gain control to amplify the electrodes used for EEG (FIG. 2), a digital signal processing (DSP) microcontroller with analog-to-digital converters (ADCs) to extract frequency information from the sampled data (FIG. 3), digital-to-analog converters (DACs) to create a software-definable alternating voltage, and a voltage-controlled-current-source which connects to the stimulation electrodes (FIG. 4). The device can download recorded EEG data to a computer via its universal serial bus (USB) interface (or, in another instantiation, a wireless interface), for further review by investigators, doctors, or health care providers.

FIG. 2 depicts an exemplary embodiment of the EEG input amplification stage. In this embodiment, gold-plated electrodes placed on the scalp (connected to terminals on X2) read potentials indicating brain activity. These signals are on the order of a few microvolts, and thus must be amplified in order for the ADCs to read the data with the required resolution. A software-adjustable potentiometer allows for automatic gain control. The amplified outputs are tied in to the built-in ADCs on the microcontroller.

FIG. 3 depicts an exemplary embodiment of the circuit for integrating EEG readout with TCS control. A microcontroller, for example, but not limited to, a PIC18 or dsPIC33F, with built-in EEPROM memory and USB functionality controls the stimulation phase and is responsible for interpreting EEG data. In the preferred embodiment, the control software resides on the DSP microcontroller and is responsible for executing a Fast Fourier Transform (FFT) on EEG data to extract the amplitude of oscillations at different frequencies (0-200 Hz). The device uses a learning algorithm that dynamically adjusts its pre-initialized sleep-cycle-prediction-vectors, to match the individual as well as to take into account any structure observed in the entire population. These vectors are used along with the data obtained by the FFT to predict the user's current sleep state. The microcontroller computes, through an algorithm, current or voltage signals to be delivered through the stimulation electrodes on the scalp. The stimulation electrodes are then activated during preset sleep states, to output 1 Hz sinusoids of 0.5 mA/cm² onto two frontal electrodes.

FIG. 4 depicts an exemplary embodiment of the current-driver output phase. Software-defined oscillations are sent as binary data to a DAC that converts the data to a voltage level. Operational amplifiers employ feedback to act as voltage-controlled-current-sources. R36 and R31 regulate the maximum amount of current delivered to the user for a given input voltage. Current delivery is limited to a one milliamp maximum for safety.

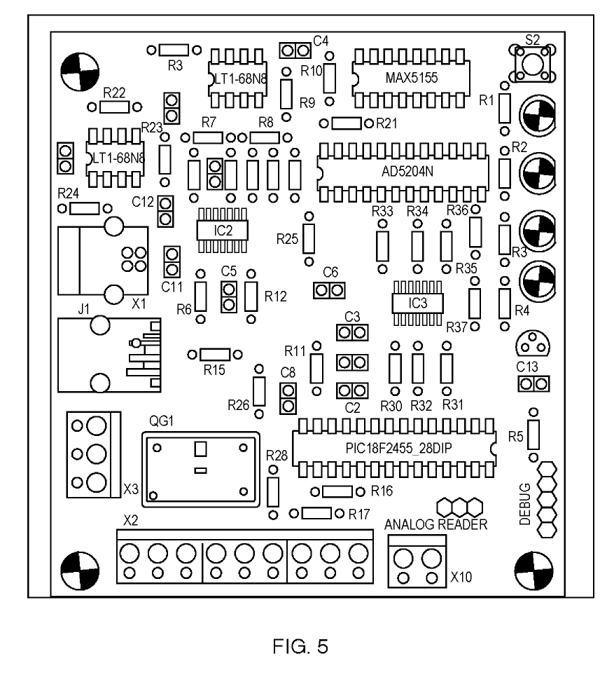

FIG. 5 depicts an exemplary board layout of an example embodiment of the present invention. In a preferred embodiment, the device is 3.5 inches square. Various indicator LEDs notify the user of the system's current state. A USB port is used for computer interfacing, permitting the EEG data to be separately analyzed by the user. Various terminal strips allow electrodes to be attached to the system.

The prototype implementation is adapted to record from 1-4 EEG electrodes, to do spectral analysis of EEG signals between 0 and 200 Hz, and to deliver 1 Hz oscillatory current amplitude from two frontal electrodes (with two mastoid electrodes providing the current return path) at the onset of the first epoch of slow wave sleep during a sleep episode. While specific devices, settings, ranges, and time intervals are employed in the current implementation, it will be clear to one of ordinary skill in the art that many other devices, settings, ranges, and time interval are suitable for use with the present invention and may be advantageously employed therein. For example, the present invention may advantageously employ more, or fewer, EEG electrodes or more signals in addition to EEG, such as, but not limited to, galvanic skin response (GSR) and infrared (IR) observation. The device can trigger on different spectral signatures of the EEG (such as alpha, delta, gamma, or other spectral signatures, the onset of REM, or intermediate sleep). The device can deliver various frequencies such as, but not limited to, gamma (for attention, or for treatment of schizophrenia) or REM broadband (for motor memory enhancement). The device can also entrain brain rhythms, dynamically delivering signals to entrain brain oscillations to a particular phase or frequency. Finally, moving beyond oscillations, the device can advantageously deliver pulsatile or other current signals, distributed across multiple electrodes on the scalp, to coordinate or disrupt oscillations as need be.

In the preferred embodiment, the entire device is battery powered and small enough to fit in a pocket, so it is wearable. Alternatively, the device can be remotely powered, e.g. by a RF inductive coil/antenna. In the smallest form, one or more button containing electrodes and integrated electronics can be placed on the scalp. In a larger form, a cohesive pattern of electrodes can be placed at multiple sites on the scalp.

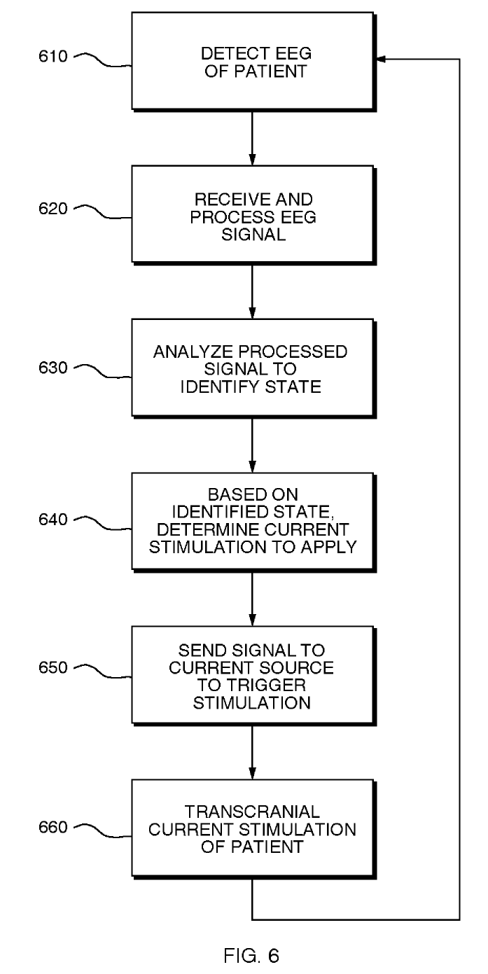

FIG. 6 is a flowchart illustrating the operation of an embodiment of the present invention. In FIG. 6, an EEG signal is detected 610, received and processed 620 and then analyzed 630 to identify the patient state. Based on the patient state, a type and amount of current stimulation to apply to the patient is determined 640 and a signal is sent 650 to the current source in order to trigger transcranial current stimulation 660 of the patient.

The present invention is therefore a “closed-loop” brain prosthetic. The closing of the loop between the EEG and TCS functions enables many different protocols. In a preferred embodiment, the EEG is linear filtered (for example, but not limited to, lowpass, high pass, bandpass and phase shift), in order to emphasize a desired EEG feature (e.g., a certain frequency) or a certain pattern of activity. This filtered version is then sent back to the patient through the TCS, thus amplifying that specific frequency in the brain. In this way, the frequencies amplified to the patient are customized to, for example, but not limited to, amplify waves that occur during sleep or attention or that do not occur in conjunction with epilepsy or Parkinson's disease. Among other benefits, this permits augmentation of specific brain functions.

A specific implementation of this type of filter-customized computation, wherein it is desired to play back a subset of signals, or a delayed or amplified set of signals, to the brain, is accomplished by the steps of: (A) measuring a block of EEG signals lasting t seconds; (B) computing a linear filter of that block of EEG signals; (C) playing back the signal through the TCS circuit; and (D) analyzing the next t seconds or, alternatively, a block lasting t seconds that overlaps with the previous block by a pre-defined window.

Another protocol useful on the platform is a phase-estimator algorithm, which permits amplification of brain waves with real-time control. In a preferred embodiment, this comprises the steps of: (A) measuring the EEG signal; (B) estimating the instantaneous phase of all the different frequency components (i.e., Fourier transform the signal and then measure all the phases of the different frequency components); (C) applying for a short period of time, using the TCS circuitry, a signal that continues each frequency at the correct phase; and (D) re-measuring the EEG signal and extract the phases again, repeating this algorithm.

It is well-known that subjects' brainwaves differ in frequency content from one another, which can limit the efficacy of current stimulation of the brain. In one embodiment of the present invention, a protocol is employed to provide patient-customized bandwidth, comprising the following steps: (A) Measuring the specific frequency bands of slow-waves, theta waves, alpha waves, beta waves, and gamma waves within a subject. This can be done by taking the Fourier transform and looking for peaks. The lowest is slow-wave, the next highest is theta, the next highest is alpha, and so on. (B) Measuring the EEG and determining what frequency bands are present. (C) Playing back the subject-specific currents to fit within the frequency bands of the subject. In this way the brain stimulation naturally entrains the individual patient's brain, rather than inducing an artificial set of frequencies.

On occasion, it is desirable to continuously entrain the brain's oscillations, for maximal efficacy. In one embodiment, this is accomplished through multiplex reading from and writing to the brain by the steps of: (A) measuring the EEG signal for t1 seconds; (B) breaking down the signal into Fourier components with amplitude and phase; (C) extrapolating, for each frequency, the signal for t2 seconds into the future; (D) playing back the signal through the TCS module for t2 seconds; and (E) repeating, perhaps with a window of t1 seconds that overlaps with the previous block by a pre-defined window.

A useful application of the present invention includes detection of REM sleep in order to amplify motor memory by delivery of current oscillations in the REM bandwidth. The device can, for example, be used to enhance motor memory via delivery of 20-60 Hz oscillatory currents, or signals that predominantly possess frequencies of 20-60 Hz. Another use of the device is to enhance declarative memory via delivery of 0-4 Hz oscillatory currents, or signals that predominantly possess frequencies of 0-4 Hz. Further, the slow-wave sleep device may be used in waking humans who are sleepy, in order to induce sleep or to simulate some of the effects of sleep in the waking human.

The device of the present invention may also be used to disrupt epileptic seizures by entraining circuits at lower frequencies than supported by the epileptic neural circuit. The device may be used to desynchronize brain activity, as in the case of an epileptic patient where oversynchronization has occurred. In one implementation, multiple electrodes are differently driven to insure that the local areas are desynchronized.

The device may also be used in one hemisphere at a time, in order to support sleep enhancement of one hemisphere at a time, akin to dolphin sleep. The device may be used to synchronize activity on a single brain (e.g., cerebral) hemisphere or to synchronize activity between multiple brain (e.g., cerebral) areas, including, but not limited to the case where the two areas are homologous and interhemispheric, the case where the two areas are functionally linked (e.g., by fMRI) and the case where the two areas are separated by a lesion (e.g., due to stroke or traumatic brain injury). It may be used to provide gamma-band currents to synchronize cortical activity, in order to act as a therapy for schizophrenic patients or to enhance attention in normal subjects.

While a preferred embodiment is disclosed, many other implementations will occur to one of ordinary skill in the art and are all within the scope of the invention. Each of the various embodiments described above may be combined with other described embodiments in order to provide multiple features. Furthermore, while the foregoing describes a number of separate embodiments of the apparatus and method of the present invention, what has been described herein is merely illustrative of the application of the principles of the present invention. Other arrangements, methods, modifications, and substitutions by one of ordinary skill in the art are therefore also considered to be within the scope of the present invention, which is not to be limited except by the claims that follow.

What is claimed is:

1. An integrated transcranial current stimulation and electroencephalography system, comprising:
at least one electrode;
a current source for providing stimulation current to the electrode; and
a microcontroller adapted to:
receive electroencephalography data from the electrode;
analyze the received data to determine a stimulation current to be delivered by the electrode; and
send a signal to the current source to induce the electrode to deliver the determined stimulation current.

2. The device of claim 1, further comprising at least a second electrode, wherein the detection and stimulation functions are performed by different electrodes.

3. The device of claim 1, further comprising at least one amplifier for amplifying signals transmitted between the electrode and the microcontroller.

4. The device of claim 2, further comprising at least one amplifier for amplifying signals transmitted between at least one electrode and the microcontroller.

5. The device of claim 1, further comprising a connection to a computer for downloading the electroencephalography data for additional analysis.

6. The device of claim 1, further comprising a connection to a computer for downloading the electroencephalography data for additional analysis.

7. An integrated transcranial current stimulation and electroencephalography device, comprising:
input for receiving electroencephalography data signals;
microcontroller, connected to the input, comprising:
electroencephalography data signal processor;
electroencephalography data signal analyzer; and
current stimulus control signal generator; and
output, connected to the microcontroller, for transmitting current stimulus control signals to a current source.

8. The device of claim 7, further comprising an interface for downloading electroencephalography data to a computer.

9. A method for integrated transcranial current stimulation and electroencephalography, comprising:
detecting electroencephalography signals from patient; and
in a single microcontroller, performing the steps of:
receiving and processing the electroencephalography signals with a microcontroller;
analyzing the processed signals to identify a patient state;
based on the identified patient state, determining the type and amount of transcranial current stimulation to apply to patient; and
sending a control signal to a current source to trigger the determined transcranial current stimulation of the patient.

10. The method of claim 9, further comprising the step of downloading the received and processed electroencephalography signals to a computer for additional analysis.

11. The method of claim 9, wherein the step of determining the type and amount of current stimulation to apply selects between precomputed control signals depending on the identified patient state.

12. The method of claim 9, wherein the step of determining the type and amount of current stimulation to apply adaptively computes the control signal depending on the identified patient state.

13. The method of claim 9, wherein the control signal causes delivery of rhythmic transcranial current stimulation to the patient.

14. The method of claim 9, wherein the control signal is sent when the identified patient state is a predetermined sleep cycle.

15. The method of claim 9, wherein the step of analyzing comprises linear filtering the electroencephalography signals to obtain a filtered signal and the step of determining the type and amount of current stimulation to apply computes the control signal based on the filtered signal.

16. The method of claim 9, wherein the control signal is computed based on a phase-estimator algorithm, comprising the steps of:
(a) measuring the received electroencephalography signal;
(b) estimating the instantaneous phase of all the different frequency components;
(c) applying, for a short period of time, a control signal that continues each frequency at the correct phase; and
(d) repeating steps (a)-(d).

17. The method of claim 9, wherein the control signal is computed to provide patient-customized bandwidth by the steps of:
measuring the specific frequency bands of each wave type for a patient;
determining which frequency bands are present in the received electroencephalography signal; and
computing a control signal designed to fit within the frequency bands of the patient.

18. The method of claim 9, wherein the control signal is computed to continuously entrain the patient's brain oscillations by multiplex reading from and writing to the brain.
